(19) United States
(12) Patent Application Publication
(10) Pub. No.: US 2022/0023216 A1
(43) Pub. Date: Jan. 27, 2022

(54) INSTANTLY SOLUBLE PARTICLE AND METHOD FOR PRODUCING THE SAME

(71) Applicant: RICOH COMPANY, LTD., Tokyo (JP)

(72) Inventors: Tatsuru MORITANI, Kanagawa (JP); Tadahiko MORINAGA, Kanagawa (JP); Yuichi SATO, Kanagawa (JP)

(21) Appl. No.: 17/426,864

(22) PCT Filed: Jun. 25, 2020
(86) PCT No.: PCT/JP2020/025026

§ 371 Date: Jul. 29, 2021

(30) Foreign Application Priority Data
Jun. 26, 2019 (JP) 2019-118289
Jun. 3, 2020 (JP) 2020-096960

(51) Int. Cl. A61K 9/14; A61K 47/26; A61K 31/517; A61K 31/5377; A61K 38/13; A61K 31/506; A61K 31/192

(52) U.S. Cl. CPC A61K 9/14; A61K 47/26; A61K 31/517; A61K 31/192; A61K 38/13; A61K 31/506; A61K 31/5377

(57) ABSTRACT

An instantly soluble particle including: a water-soluble base material; and a poorly water-soluble compound, wherein the water-soluble base material contains a rapidly water-soluble compound, and the poorly water-soluble compound exists in the water-soluble base material in an amorphous state.

### TECHNICAL FIELD

The present disclosure relates to an instantly soluble particle and a method for producing the instantly soluble particle.

### BACKGROUND ART

Currently, compounds that have been newly developed as medicines include many compounds having a considerably poor solubility in water, so-called poorly water-soluble compounds.

When such a poorly water-soluble compound is orally administered, the poorly water-soluble compound as a medicine is not sufficiently dissolved in the body, which may result in a decrease in bioavailability. In order to avoid the decrease in bioavailability, various approaches to dissolve the poorly water-soluble compound have been performed. For example, improvement in a dissolution rate achieved by forming a pharmaceutical agent that is the poorly water-soluble compound into nanoparticles to increase a surface area of the particles of the pharmaceutical agent and use of a solubilizer exhibiting solubilization in the pharmaceutical agent in combination have been investigated. Particularly, many particles improved in solubility, which are obtained by introducing a pharmaceutical agent into an inert base material, have been investigated.

For example, a solid pharmaceutical preparation that instantly releases a pharmaceutical compound with a low solubility by incorporating the pharmaceutical compound dissolved in a solubilizer into the solid pharmaceutical preparation has been proposed (see, for example, PTL 1).

A solid dispersing element improved in solubility of a poorly water-soluble compound obtained by including, for example, a water-soluble polymer, a water-soluble saccharide, and a surfactant has been proposed (see, for example, PTL 2). As described above, various approaches to improve solubility of a poorly water-soluble pharmaceutical compound have been performed.

### CITATION LIST

### Patent Literature

PTL 1: Japanese Patent No. 2960169

PTL 2: Japanese Patent No. 5484910

### SUMMARY OF INVENTION

### Technical Problem

An object of the present disclosure is to provide an instantly soluble particle that can rapidly dissolve a poorly water-soluble compound.

### Solution to Problem

According to one aspect of the present disclosure, an instantly soluble particle includes a water-soluble base material and a poorly water-soluble compound. The water-soluble base material contains a rapidly water-soluble compound. The poorly water-soluble compound exists in an amorphous state in the water-soluble base material.

### Advantageous Effects of Invention

According to the present disclosure, it is possible to provide an instantly soluble particle that can rapidly dissolve a poorly water-soluble compound.

### BRIEF DESCRIPTION OF DRAWINGS

FIG. 1 is a cross-sectional view presenting one example of a liquid droplet formation unit.

FIG. 2 is a cross-sectional view presenting one example of a liquid column resonance droplet-discharging unit.

FIG. 3A is a schematic view presenting one example of a structure of a discharging hole.

FIG. 3B is a schematic view presenting another example of a structure of a discharging hole.

FIG. 3C is a schematic view presenting another example of a structure of a discharging hole.

FIG. 3D is a schematic view presenting another example of a structure of a discharging hole.

FIG. 4A is a schematic view presenting a standing wave of velocity fluctuation and a standing wave of pressure fluctuation when N=1 and one end is fixed.

FIG. 4B is a schematic view presenting a standing wave of velocity fluctuation and a standing wave of pressure fluctuation when N=2 and both ends are fixed.

FIG. 4C is a schematic view presenting a standing wave of velocity fluctuation and a standing wave of pressure fluctuation when N=2 and both ends are free.

FIG. 4D is a schematic view presenting a standing wave of velocity fluctuation and a standing wave of pressure fluctuation when N=3 and one end is fixed.

FIG. 5A is a schematic view presenting a standing wave of velocity fluctuation and a standing wave of pressure fluctuation when N=4 and both ends are fixed.

FIG. 5B is a schematic view presenting a standing wave of velocity fluctuation and a standing wave of pressure fluctuation when N=4 and both ends are free.

FIG. 5C is a schematic view presenting a standing wave of velocity fluctuation and a standing wave of pressure fluctuation when N=5 and one end is fixed.

FIG. 6A is a schematic view presenting one exemplary pressure and velocity waveforms in a liquid-column-resonance liquid chamber when liquid droplets are discharged.

FIG. 6B is a schematic view presenting another exemplary pressure and velocity waveforms in a liquid-column-resonance liquid chamber when liquid droplets are discharged.

FIG. 6C is a schematic view presenting another exemplary pressure and velocity waveforms in a liquid-column-resonance liquid chamber when liquid droplets are discharged.

FIG. 6D is a schematic view presenting another exemplary pressure and velocity waveforms in a liquid-column-resonance liquid chamber when liquid droplets are discharged.

FIG. 6E is a schematic view presenting another exemplary pressure and velocity waveforms in a liquid-column-resonance liquid chamber when liquid droplets are discharged.

FIG. 7 is an image presenting exemplary actual liquid droplets discharged by a liquid droplet formation unit.

FIG. 8 is a graph presenting dependency of a liquid droplet-discharging velocity on a driving frequency.

FIG. 9 is a schematic view presenting one exemplary particle production apparatus.

FIG. 10 is a schematic view presenting one exemplary gas flow path.

FIG. 11 is a graph presenting results of the dissolution test of Test Example 1.

FIG. 12 is a graph presenting results of the dissolution test of Test Example 2.

FIG. 13 is a graph presenting results of the dissolution test of Test Example 3.

FIG. 14 is an X-ray diffraction pattern of the pharmaceutical agent used in Example 11.

FIG. 15 is an X-ray diffraction pattern of the particle prepared in Example 11.

### DESCRIPTION OF EMBODIMENTS

(Instantly Soluble Particle)

An instantly soluble particle of the present disclosure includes a water-soluble base material and a poorly water-soluble compound. The water-soluble base material contains a rapidly water-soluble compound. The poorly water-soluble compound exists in the water-soluble base material in an amorphous state. The instantly soluble particle further includes other ingredients if necessary.

The instantly soluble particle of the present disclosure can be suitably produced by a method of the present disclosure for producing the instantly soluble particle that will be described hereinafter.

In the present disclosure, the term “instantly soluble particle” means a particle, which includes a poorly water-soluble compound and can be rapidly dissolved in water when added to water to form a solution or a dispersion liquid of the poorly water-soluble compound. The “being rapidly dissolved” or the “instantly soluble” may be different depending on a size of a particle, a temperature of a solvent, and solubility of a compound, but can be evaluated by using various methods known in the art (e.g., measurement of dissolution time). One example of the specific evaluation methods is, but is not limited to, the following method. A particle to be evaluated is added to, for example, water or a physiological saline solution so that a concentration of the poorly water-soluble compound reaches a certain concentration (e.g., 1% by mass). The resultant is shaken or stirred at a constant pace (e.g., two times per second). The time taken for the particle to completely dissolve is measured. For example, such a particle that is completely dissolved to an extent that the particle cannot be visually confirmed within a certain time (e.g., within 30 minutes, within 20 minutes, within 10 minutes, within 5 minutes, within 3 minutes, within 2 minutes, within 1 minute, within 50 seconds, within 40 seconds, within 30 seconds, within 20 seconds, and within 10 seconds) is evaluated as being rapidly dissolved or being instantly soluble. When the particle is instantly soluble, it does not typically require special operation for dissolution (for example, continuous stirring over several hours, and atomization by using a homogenizer).

In the present disclosure, the “rapidly water-soluble compound” means a compound that has a property of being rapidly dissolved in water with short-time stirring or shaking within a certain time (e.g., within 1 minute, within 50 seconds, within 40 seconds, within 30 seconds, within 20 seconds, and within 10 seconds) without performing special operation for dissolution when the rapidly water-soluble compound is added to water. Generally, there is a tendency that it takes a longer time for a water-soluble compound to dissolve as the molecular weight thereof increases. Therefore, the rapidly water-soluble compound is preferably a low-molecular-weight water-soluble compound having a molecular weight of less than 10,000. Examples of the rapidly water-soluble compound include, but are not limited to, low-molecular saccharides (e.g., monosaccharides and disaccharides), oligosaccharides, reducing sugars, and sugar alcohols. The rapidly water-soluble compound that can be used in the present disclosure is preferably a solid at normal temperature.

As a result of diligent studies to rapidly dissolve a compound exhibiting a poor water-solubility, the present inventors obtained the following finding. Specifically, when a microparticle containing a poorly water-soluble compound is produced by using, as a base material, a substance (e.g., monosaccharide and disaccharide) that is rapidly dissolved in water, the microparticle is rapidly dissolved in water to form an aqueous solution of the poorly water-soluble compound.

The instantly soluble particle of the present disclosure is in an amorphous state. Without wishing to be bound by any theory, it is understood that the instantly soluble particle of the present disclosure has a solid dispersion structure in which an amorphous poorly water-soluble compound is dispersed in an amorphous water-soluble base material. Therefore, when the instantly soluble particle of the present application is added to water, the surrounding water-soluble base material is rapidly dissolved to thereby rapidly disperse, in water, the poorly water-soluble compound in a dispersed state. Moreover, it is deemed that because both the water-soluble base material and the poorly water-soluble compound are amorphous, the water-soluble base material and the poorly water-soluble compound in an amorphous state are more energetically unstable than those in a crystalline state, and thus rapid dissolution in water can be achieved.

—Water-Soluble Base Material—

The water-soluble base material is not particularly limited so long as the water-soluble base material itself is rapidly dissolved in water and can be dispersed in a base material without chemically reacting with the poorly water-soluble compound. Examples of the water-soluble base material include rapidly water-soluble compounds.

Examples of the rapidly water-soluble compound include monosaccharides, disaccharides, oligosaccharides, reducing sugars, and sugar alcohols.

Examples of the monosaccharide include glucose, mannose, idose, galactose, fucose, ribose, and xylose.

Examples of the disaccharide include lactose, sucrose, maltose, and trehalose.

Examples of the oligosaccharide include raffinose (trisaccharide), maltotriose (trisaccharide), and acarbose (tetrasaccharide).

Examples of the reducing sugar include turanose.

Examples of the sugar alcohol include glycerin, erythritol, xylitol, lactitol, sorbitol, and maltitol.

When the instantly soluble particle of the present disclosure includes the water-soluble base material, it is possible to improve solubility of a poorly water-soluble compound that will be described below in water and to improve wettability of the instantly soluble particle to a solvent when the instantly soluble particle is dissolved in a solution and wettability of the poorly water-soluble compound contained in the instantly soluble particle.

An amount of the water-soluble base material in the instantly soluble particle is not particularly limited and may be appropriately selected depending on the intended purpose, so long as the amount of the water-soluble base material is such an amount that a function of rapidly dissolving the poorly water-soluble compound in water can be exhibited. The amount of the water-soluble base material is preferably 30% by mass or more but 80% by mass or less, more preferably 50% by mass or more but 80% by mass or less.

—Poorly Water-Soluble Compound—

The poorly water-soluble compound means a compound having a water/octanol partition coefficient (log P value) of 3 or more.

The water/octanol partition coefficient means a ratio between a concentration of a compound dissolved in an aqueous phase and a concentration of the compound dissolved in an octanol phase in a two-phase system of water and octanol, and is generally represented by Log₁₀ (concentration of compound in octanol phase/concentration of compound in aqueous phase).

A method for measuring the water/octanol partition coefficient (log P value) can be any known method in the art. Examples of the method include the method described in JIS Z 7260-107.

The poorly water-soluble compound is not particularly limited and may be appropriately selected depending on the intended purpose, so long as it has a water/octanol partition coefficient (log P value) of 3 or more. Examples of the poorly water-soluble compound include physiologically active substances.

The physiologically active substance is not particularly limited and may be appropriately selected depending on the intended purpose. Examples of the physiologically active substance include pharmaceutical compounds, cosmetic compounds, and functional food compounds. Pharmaceutical compounds are preferable. The pharmaceutical compound may be any compound known as an active ingredient of a medicament. Examples of the pharmaceutical compound include low-molecular pharmaceutical compounds, kinase inhibitors (e.g., tyrosine kinase inhibitor and serine/threonine kinase inhibitor), polypeptides, and nucleic acids (e.g., antisense nucleic acid).

Examples of the kinase inhibitor include gefitinib, erlotinib, osimertinib, bosutinib, vandetanib, alectinib, lorlatinib, abemaciclib, tyrphostin AG494, sorafenib, dasatinib, lapatinib, imatinib, motesanib, lestaurtinib, tandutinib, dorsomorphin, axitinib, 4-benzyl-2-methyl-1,2,4-thiadiazolidine-3,5-dione, and afatinib.

Examples of the polypeptide include ciclosporin, vancomycin, teicoplanin, and daptomycin.

Examples of other poorly water-soluble compounds include quercetin, testosterone, indomethacin, tranilast, tacrolimus, and ibuprofen. Among them, kinase inhibitors and polypeptides are preferable.

An amount of the poorly water-soluble compound in the instantly soluble particle is not particularly limited and may be appropriately selected depending on the intended purpose, so long as an effect of the poorly water-soluble compound can be sufficiently achieved and the amount falls within a range where the poorly water-soluble compound can be dissolved in water. As the amount of the poorly water-soluble compound increases, the solubility decreases accordingly. In one embodiment, the amount of the poorly water-soluble compound can be, for example, 75% by mass or less, preferably 1% by mass or more but 75% by mass or less, more preferably 10% by mass or more but 70% by mass or less, still more preferably 20% by mass or more but 50% by mass or less. When the amount of the poorly water-soluble compound is 1% by mass or more, an amount of a solution of the instantly soluble particle required to administer a required amount of a pharmaceutical agent can be decreased. Meanwhile, the amount of the poorly water-soluble compound is 75% by mass or less, it is possible to bear a high instant solubility of the poorly water-soluble compound. When the amount of the poorly water-soluble compound is 1% by mass or less, a concentration of the pharmaceutical agent is decreased at the time of taking the pharmaceutical agent. As a result, a larger amount of the solution should be administered, which is not efficient. Meanwhile, the amount of the poorly water-soluble compound is 75% by mass or more, an effect of instant solubility of the pharmaceutical agent is decreased.

As described above, the instantly soluble particle of the present disclosure in one embodiment includes a pharmaceutical compound, and is particularly in the form where a poorly soluble pharmaceutical compound can be rapidly dissolved in water. Therefore, the particle of the present disclosure can be particularly suitably used in a dosage form of a pharmaceutical composition that is used by being dissolved in water. In addition, it can be suitably used as a pharmaceutical composition that can be prepared at the time of use.

A volume average particle diameter (Dv) of the instantly soluble particle is preferably 0.5 μm or more but 50 μm or less, more preferably 0.5 μm or more but 20 m or less. When the volume average particle diameter (Dv) of the instantly soluble particle is 0.5 μm or more but 50 μm or less, the poorly water-soluble compound contained in the instantly soluble particle is easily included in the particle in an amorphous state, which increases solubility of the poorly water-soluble compound.

In one embodiment, the instantly soluble particle has a relative span factor (R.S.F) that satisfies the following expression (1).

0<(R.S.F)≤1.5 Expression (1)

The (R.S.F) is defined as (D90−D10)/D50.

The D90 denotes a cumulative 90% by volume from a small particle side of a cumulative particle size distribution, the D50 denotes a cumulative 50% by volume from the small particle side of the cumulative particle size distribution, and the D10 denotes a cumulative 10% by volume from the small particle side of the cumulative particle size distribution. The upper limit of the R.S.F. is not particularly limited. Examples of the upper limit include 1.5, 1.4, 1.3, 1.2, 1.1, 1.0, 0.9, 0.8, 0.7, 0.6, and 0.5.

The (R.S.F) can be measured by, for example, a laser diffraction/scattering particle size distribution analyzer (device name: MICROTRAC MT3000II, available from MicrotracBEL Corp.) or a fiber-optics particle analyzer (“FPAR-1000”, available from Otsuka Electronics Co., Ltd.) using the dynamic light scattering method.

—Other Ingredients—

The other ingredients are not particularly limited and may be appropriately selected depending on the intended purpose.

(Method for producing instantly soluble particle and apparatus for producing instantly soluble particle) A method of the present disclosure for producing an instantly soluble particle includes: discharging a liquid containing a rapidly water-soluble compound and a poorly water-soluble compound from a discharging hole to form liquid droplets; and solidifying the liquid droplets. The method of the present disclosure for producing an instantly soluble particle further includes other steps if necessary.

An apparatus of the present disclosure for producing an instantly soluble particle includes: a liquid droplet formation unit configured to discharge a liquid containing a rapidly water-soluble compound and a poorly water-soluble compound from a discharging hole to form liquid droplets; and a liquid droplet solidifying unit configured to solidify the liquid droplets. The apparatus of the present disclosure for producing an instantly soluble particle further includes a solidified particle collecting unit and other units if necessary.

<Liquid Droplet Forming Step and Liquid Droplet Formation Unit>

The liquid droplet forming step is a step of discharging a liquid containing the rapidly water-soluble compound and the poorly water-soluble compound from a discharging hole to form liquid droplets, and is performed by the liquid droplet formation unit.

—Liquid—

The liquid includes the water-soluble base material and the poorly water-soluble compound in a solvent, and further includes other ingredients if necessary.

——Solvent——

The solvent is not particularly limited and may be appropriately selected depending on the intended purpose. Preferable examples of the solvent include those that can dissolve or disperse the water-soluble base material and the poorly water-soluble compound, or a pharmaceutical acceptable salt thereof. In order to simultaneously dissolve the water-soluble base material and the poorly water-soluble compound, two or more kinds of solvents are preferably mixed for use.

Examples of the solvent include water, aliphatic halogenated hydrocarbons (e.g., dichloromethane, dichloroethane, and chloroform), alcohols (e.g., methanol, ethanol, and propanol), ketones (e.g., acetone and methyl ethyl ketone), ethers (e.g., diethyl ether, dibutyl ether, and 1,4-dioxane), aliphatic hydrocarbons (e.g., n-hexane, cyclohexane, and n-heptane), aromatic hydrocarbons (e.g., benzene, toluene, and xylene), organic acids (e.g., acetic acid and propionic acid), esters (e.g., ethyl acetate), amides (e.g., dimethylformamide and dimethylacetamide), and mixture solvents thereof.

An amount of the solvent is preferably 70% by mass or more but 99.5% by mass or less, more preferably 90% by mass or more but 99% by mass or less, relative to a total amount of the liquid. When the amount of the solvent is 70% by mass or more but 99.5% by mass or less relative to the total amount of the liquid, production stability can be improved because solubility of the poorly water-soluble compound and viscosity of the liquid can be appropriate.

——Rapidly Water-Soluble Compound——

The rapidly water-soluble compound is the same that can be used in the instantly soluble particle of the present disclosure.

An amount of the rapidly water-soluble compound is preferably 0.1% by mass or more but 20.0% by mass or less, more preferably 0.1% by mass or more but 15.0% by mass or less, relative to the total amount of the liquid.

——Poorly Water-Soluble Compound——

The poorly water-soluble compound is the same that can be used in the instantly soluble particle of the present disclosure.

An amount of the poorly water-soluble compound is preferably 0.05% by mass or more but 5.0% by mass or less, more preferably 0.1% by mass or more but 3.0% by mass or less, relative to the total amount of the liquid.

——Other Ingredients——

The other ingredients are not particularly limited and may be appropriately selected depending on the intended purpose. They are preferably those that can conventionally be used in medicaments.

Examples of the other ingredients include water, an excipient, a flavoring agent, a disintegrating agent, a fluidizer, an adsorbent, a lubricant, an odor-masking agent, a surfactant, a perfume, a colorant, an anti-oxidant, a masking agent, an anti-static agent, and a humectant. These may be used alone or in combination.

The excipient is not particularly limited and may be appropriately selected depending on the intended purpose. Examples of the excipient include lactose, sucrose, mannitol, glucose, fructose, maltose, erythritol, maltitol, xylitol, palatinose, trehalose, sorbitol, crystalline cellulose, talc, silicic anhydride, anhydrous calcium phosphate, precipitated calcium carbonate, and calcium silicate. These may be used alone or in combination.

The flavoring agent is not particularly limited and may be appropriately selected depending on the intended purpose. Examples of the flavoring agent include L-menthol, sucrose, D-sorbitol, xylitol, citric acid, ascorbic acid, tartaric acid, malic acid, aspartame, acesulfame potassium, thaumatin, saccharin sodium, dipotassium glycyrrhizate, sodium glutamate, sodium 5′-inosinate, and sodium 5′-guanylate. These may be used alone or in combination.

The disintegrating agent is not particularly limited and may be appropriately selected depending on the intended purpose. Examples of the disintegrating agent include low-substituted hydroxypropylcellulose, carmellose, carmellose calcium, carboxymethyl starch sodium, croscarmellose sodium, crospovidone, hydroxypropyl starch, and corn starch. These may be used alone or in combination.

The fluidizer is not particularly limited and may be appropriately selected depending on the intended purpose. Examples of the fluidizer include light anhydrous silicic acid, hydrated silicon dioxide, and talc. These may be used alone or in combination.

As the light anhydrous silicic acid, a commercially available product can be used. The commercially available product of light anhydrous silicic acid is not particularly limited and may be appropriately selected depending on the intended purpose. Examples of the commercially available product of light anhydrous silicic acid include ADSOLIDER 101 (available from Freund Corporation: average pore diameter: 21 nm).

As the adsorbent, a commercially available product can be used. The commercially product of the adsorbent is not particularly limited and may be appropriately selected depending on the intended purpose. Examples of the commercially product of the adsorbent include product name: CARPLEX (ingredient name: synthetic silica, registered trademark of Evonik Japan), product name: AEROSIL (registered trademark of NIPPON AEROSIL CO., LTD.) 200 (ingredient name: hydrophilic fumed silica), product name: SYLYSIA (ingredient name: amorphous silicon dioxide, registered trademark of Fuji Silysia chemical Ltd.), and product name: ALCAMAC (ingredient name: synthetic hydrotalcite, registered trademark of Kyowa Chemical Industry Co., Ltd.). These may be used alone or in combination.

The lubricant is not particularly limited and may be appropriately selected depending on the intended purpose. Examples of the lubricant include magnesium stearate, calcium stearate, sucrose fatty acid ester, sodium stearyl fumarate, stearic acid, polyethylene glycol, and talc. These may be used alone or in combination.

The odor-masking agent is not particularly limited and may be appropriately selected depending on the intended purpose. Examples of the odor-masking agent include trehalose, malic acid, maltose, potassium gluconate, anise essential oil, vanilla essential oil, and cardamom essential oil. These may be used alone or in combination.

The surfactant is not particularly limited and may be appropriately selected depending on the intended purpose. Examples of the surfactant include Polysorbates (e.g., Polysorbate 80); polyoxyethylene⋅polyoxypropylene copolymer; and sodium lauryl sulfate. These may be used alone or in combination.

The perfume is not particularly limited and may be appropriately selected depending on the intended purpose. Examples of the perfume include lemon oil, orange oil, and peppermint oil. These may be used alone or in combination.

The colorant is not particularly limited and may be appropriately selected depending on the intended purpose. Examples of the colorant include titanium oxide, Food Yellow No. 5, Food Blue No. 2, Ferric oxide, and Yellow Ferric Oxide. These may be used alone or in combination.

The anti-oxidant is not particularly limited and may be appropriately selected depending on the intended purpose. Examples of the anti-oxidant include sodium ascorbate, L-cysteine, sodium sulfite, and vitamin E. These may be used alone or in combination.

The masking agent is not particularly limited and may be appropriately selected depending on the intended purpose. Examples of the masking agent include titanium oxide. These may be used alone or in combination.

The anti-static agent is not particularly limited and may be appropriately selected depending on the intended purpose. Examples of the anti-static agent include talc and titanium oxide. These may be used alone or in combination.

The humectant is not particularly limited and may be appropriately selected depending on the intended purpose. Examples of the humectant include Polysorbate 80, sodium lauryl sulfate, sucrose fatty acid ester, macrogol, and hydroxypropylcellulose (HPC). These may be used alone or in combination.

The liquid may not include a solvent so long as the liquid is in a state that the water-soluble base material and the poorly water-soluble compound are dissolved, the liquid is in a state that the poorly water-soluble compound is dispersed, or the liquid is a liquid when discharged. The liquid may be in a state that a particle ingredient is melted.

—Discharging Hole—

The discharging hole is not particularly limited and may be appropriately selected depending on the intended purpose. Examples of the discharging hole include an opening provided in, for example, a nozzle plate.

The number, a cross-sectional shape, and a size of the discharging holes can be appropriately selected.

The number of discharging holes is not particularly limited and may be appropriately selected depending on the intended purpose. For example, the number thereof is preferably 2 or more but 3,000 or less. When the number of discharging holes is 2 or more but 3,000 or less, productivity can be improved.

A cross-sectional shape of the discharging hole is not particularly limited and may be appropriately selected depending on the intended purpose. Examples of the cross-sectional shape include: (1) such a tapered shape that an opening diameter is decreased from a liquid contact surface (inlet) of a discharging hole toward a discharging hole (outlet); (2) such a shape that an opening diameter is narrowed from a liquid contact surface (inlet) of a discharging hole toward a discharging hole (outlet) while its round shape is maintained; (3) such a shape that an opening diameter is narrowed from a liquid contact surface (inlet) of a discharging hole toward a discharging hole (outlet) while a certain nozzle angle is maintained; and (4) combinations of the shape of (1) and the shape of (2). Among them, (3) such a shape that an opening diameter is narrowed from a liquid contact surface (inlet) of a discharging hole toward a discharging hole (outlet) while a certain nozzle angle is maintained is preferable because pressure to be applied to a liquid at the discharging hole reaches the maximum.

The nozzle angle in the shape of (3) is not particularly limited and may be appropriately selected depending on the intended purpose. The nozzle angle thereof is preferably 600 or more but 900 or less. When the nozzle angle is 600 or more, pressure is easily applied to a liquid, and processing is easily performed. When the nozzle angle is 900 or less, pressure can be applied at the discharging hole to stabilize discharging of liquid droplets. Therefore, the maximum value of the nozzle angle is preferably 90°.

A size of the discharging hole can be appropriately selected considering the sustained-releasability of a particle to be produced. For example, a diameter of the discharging hole is preferably 12 μm or more but 100 μm or less, more preferably 15 μm or more but 30 μm or less. When the size of the discharging hole is 12 μm or more but 100 μm or less, it is possible to obtain a particle having such a sufficient particle diameter that achieves sustained-releasability.

<<Liquid Droplet Formation Unit>>

The liquid droplet formation unit is not particularly limited and a known liquid droplet formation unit may be appropriately used depending on the intended purpose. Examples of the liquid droplet formation unit include spray nozzles, one-fluid nozzles, two-fluid nozzles, film vibration-type discharging units, Rayleigh-breakup-type discharging units, liquid vibration-type discharging units, and liquid column resonance-type discharging units.

Examples of the film vibration-type discharging unit include discharging units described in Japanese Unexamined Patent Application Publication No. 2008-292976. Examples of the Rayleigh-breakup-type discharging unit include discharging units described in Japanese Patent No. 4647506. Examples of the liquid vibration-type discharging unit include discharging units described in Japanese Unexamined Patent Application Publication No. 2010-102195.

In order to narrow the particle size distribution of the liquid droplet and ensure productivity of the instantly soluble particle, it is possible to employ liquid column resonance for forming liquid droplets with the liquid column resonance-type discharging unit. In the liquid column resonance for forming liquid droplets, vibration may be imparted to a liquid in a liquid-column-resonance liquid chamber to form standing waves through liquid column resonance, to discharge the liquid from a plurality of the discharging holes formed to regions that correspond to anti-nodes of the standing waves.

Examples of the liquid discharged by the liquid droplet formation unit in the present disclosure include an embodiment of a “particle ingredient-containing liquid” in which a particle ingredient to be obtained is dissolved or dispersed. The liquid may not include a solvent so long as it is a liquid when discharged, and may be an embodiment of a “particle ingredient-melted liquid” in which the particle ingredient is melted.

<Liquid Droplet Solidifying Step>

The liquid droplet solidifying step is a step of solidifying the liquid droplets.

Specifically, the liquid droplet solidifying step is a step of solidifying liquid droplets of the liquid containing the rapidly water-soluble compound and the poorly water-soluble compound discharged into a gas from the liquid droplet formation unit.

The liquid droplet solidifying unit is a unit configured to solidify the liquid droplets.

<<Liquid Droplet Solidifying Unit>>

Solidification of the liquid droplets is not particularly limited and may be appropriately selected depending on characteristics of the liquid so long as the liquid can become a solid. For example, when the liquid obtained by dissolving or dispersing a solid raw material in a volatile solvent is used, liquid droplets are discharged, and the liquid droplets are discharged into a conveyance gas flow, followed by drying. That is, solidification of the liquid droplets can be achieved by discharging the liquid droplets into the conveyance gas flow and volatilizing the solvent in the liquid droplets. In order to dry the solvent, a drying condition can be adjusted by appropriately selecting a temperature and a vapor pressure of a gas to be discharged and kinds of gases. Even when the solvent is not completely dried, additional drying may be performed in another step after collecting, so long as collected particles are kept solid. In addition, a solidification condition may be achieved through a change of temperatures or chemical reaction.

The conveyance gas flow prevents a decrease in the liquid droplet-discharging velocity immediately after the liquid droplet is discharged, and suppresses cohesion (unification) of the liquid droplets. The conveyance gas flow is provided for the following reasons.

When discharged liquid droplets contact with each other before the liquid droplets are dried, the liquid droplets are unified to form one liquid droplet (hereinafter, this phenomenon is referred to as coalescence). In order to obtain a particle having a uniform (narrow) particle size distribution, it is necessary to maintain a certain distance between the discharged droplets. However, the discharged liquid droplet travels at a certain initial velocity, but the velocity of the liquid droplet is decreased soon due to air resistance. The liquid droplet decreased in the velocity is caught up with by a liquid droplet subsequently discharged, which leads to coalescence. This phenomenon occurs regularly, and thus particle size distribution of the resultant particle is not uniform (narrow). In order to prevent coalescence of the liquid droplets, it is necessary to prevent a decrease in the liquid droplet-discharging velocity, and to solidify/convey the liquid droplet while coalescence of the liquid droplets is prevented by means of conveyance gas flow so that the liquid droplets do not contact with each other.

A method for solidifying the liquid droplet using the conveyance gas flow is not particularly limited and may be appropriately selected depending on the intended purpose. Preferable examples of the method include a method where a conveyance direction of the conveyance gas flow is a substantially vertical direction to a direction in which the liquid droplet is to be discharged. The drying method using the conveyance gas flow will be described in detail in the description of drawings that will be described hereinafter.

In order to dry the solvent, it is preferable to adjust, for example, the temperature and the vapor pressure of the conveyance gas flow, and kinds of gasses.

As long as a collected particle is kept solid, even when the collected particle is not completely dried, a drying step may be additionally provided in another step after the collecting.

In addition, a method for drying the liquid droplet by application of a temperature change or a chemical change may be used.

<Other Steps>

The other steps are not particularly limited and may be appropriately selected depending on the intended purpose. Examples of the other steps include a particle collecting step.

The particle collecting step is a step of collecting a dried particle and can be suitably performed by a particle collecting unit.

The particle collecting unit is not particularly limited and may be appropriately selected depending on the intended purpose. Examples of the article collecting unit include cyclone collection and bag filters.

A method of the present disclosure for producing an instantly soluble particle can be suitably performed by an apparatus for producing an instantly soluble particle.

Here, the apparatus for producing an instantly soluble particle will be described.

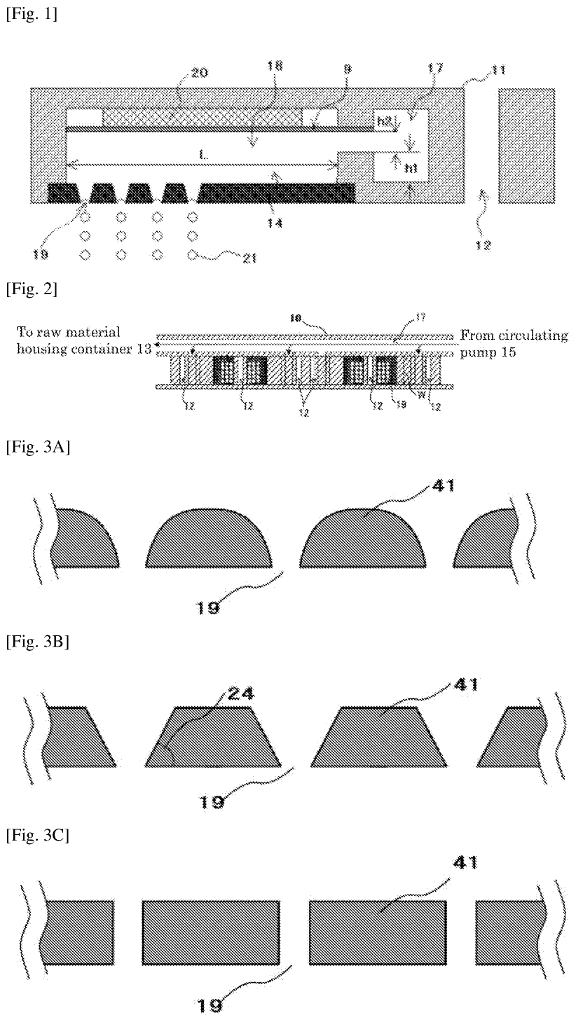

FIG. 1 is a schematic cross-sectional view of the liquid droplet formation unit 11. The liquid droplet formation unit 11 includes a common liquid supplying path 17 and a liquid-column-resonance liquid chamber 18. The liquid-column-resonance liquid chamber 18 is connected to the common liquid supplying path 17 disposed on one of wall surfaces at both side wall surfaces in a longitudinal direction. Moreover, the liquid-column-resonance liquid chamber 18 includes a discharging hole 19 and a vibration generating unit 20. The discharging hole 19 is configured to discharge liquid droplets 21, and is disposed on one wall surface of the wall surfaces connected to the side wall surfaces. The vibration generating unit 20 is configured to generate high frequency vibration to form liquid column resonance standing waves, and is disposed on the wall surface facing the discharging hole 19. Note that, a high frequency power source, which is not presented, is coupled to the vibration generating unit 20. In FIG. 1, the reference numeral 9 denotes an elastic plate, the reference numeral 12 denotes a gas flow path, and the reference numeral 14 denotes liquid.

FIG. 2 is a schematic cross-sectional view of another example of a liquid droplet formation unit. FIG. 2 presents a liquid column resonance droplet-discharging unit 10 including the liquid droplet formation unit presented in FIG. 1. The liquid 14 is allowed to pass through the liquid supplying pipe by a liquid circulating pump that is not presented to flow into the common liquid supplying path 17 of the liquid column resonance droplet-discharging unit 10 presented in FIG. 2. Then, the liquid 14 passes through the liquid supplying path of the liquid droplet formation unit 11 presented in FIG. 1 from the common liquid supplying path 17 and is supplied to the liquid-column-resonance liquid chamber 18. Within the liquid-column-resonance liquid chamber 18 charged with the liquid 14, a pressure distribution is formed by liquid column resonance standing waves generated by the vibration generating unit 20. Then, liquid droplets 21 are discharged from the discharging hole 19 disposed in the regions that correspond to anti-nodes of the standing waves and are the sections where the amplitude of the liquid column resonance standing waves is large and pressure displacement is large. The regions corresponding to anti-nodes of the standing waves owing to the liquid column resonance are regions other than nodes of the standing waves. The regions are preferably regions each having sufficiently large amplitude enough to discharge the liquid through the pressure displacement of the standing waves, more preferably regions having a width corresponding to ±¼ of a wavelength from a position of a local maximum amplitude of a pressure standing wave (i.e., a node of a velocity standing wave) toward positions of a local minimum amplitude.

Even when there are a plurality of openings of the discharging hole, substantially uniform liquid droplets can be formed from the openings as long as the openings of the discharging hole are disposed in the regions corresponding to the anti-nodes of the standing waves. Moreover, discharging of the liquid droplets can be performed efficiently, and clogging of the discharging hole is unlikely to occur. Note that, the liquid 14 passing through the common liquid supplying path 17 travels through a liquid returning pipe (not presented) to be returned to the raw material housing container. Once the amount of the liquid 14 inside the liquid-column-resonance liquid chamber 18 is reduced by discharging of the liquid droplets 21, a flow rate of the liquid 14, which is supplied from the liquid supplying path by suction power generated by the action of the liquid column resonance standing waves inside the liquid-column-resonance liquid chamber 18, is increased. As a result, the liquid-column-resonance liquid chamber 18 is refilled with the liquid 14. When the liquid-column-resonance liquid chamber 18 is refilled with the liquid 14, the flow rate of the liquid 14 passing through the liquid supplying path returns to the previous flow rate.

The liquid-column-resonance liquid chamber 18 of the liquid droplet formation unit 11 is formed by joining frames with each other. The frames are formed of materials having high stiffness to the extent that a resonance frequency of the liquid is not influenced at a driving frequency (e.g., metals, ceramics, and silicones). As presented in FIG. 1, a length L between the side wall surfaces of the liquid-column-resonance liquid chamber 18 in a longitudinal direction is determined based on the principle of the liquid column resonance described below. Moreover, a width W of the liquid-column-resonance liquid chamber 18 presented in FIG. 2 is preferably smaller than half of the length L of the liquid-column-resonance liquid chamber 18 so that excess frequency is not given to liquid column resonance. Moreover, a plurality of the liquid-column-resonance liquid chambers 18 are preferably disposed per one liquid droplet formation unit 10 in order to drastically improve productivity. The number of the liquid-column-resonance liquid chambers 18 is not particularly limited and may be appropriately selected depending on the intended purpose. The number of the liquid-column-resonance liquid chambers 18 is preferably 100 or greater but 2,000 or less in order to achieve both productivity and operability. In each liquid-column-resonance liquid chamber 18, the common liquid supplying path 17 is coupled to and connected to the liquid supplying path configured to supply the liquid. The liquid supplying path is coupled to a plurality of the liquid-column-resonance liquid chambers 18.

Moreover, the vibration generating unit 20 of the liquid droplet formation unit 11 is not particularly limited as long as the vibration generating unit 20 is driven at a predetermined frequency. The vibration generating unit is preferably formed by attaching a piezoelectric material onto an elastic plate 9. The frequency is preferably 150 kHz or greater, more preferably 300 kHz or greater but 500 kHz or less from the viewpoint of productivity. The elastic plate constitutes a portion of the wall of the liquid-column-resonance liquid chamber in a manner that the piezoelectric material does not come into contact with the liquid. The piezoelectric material may be, for example, a piezoelectric ceramic such as lead zirconate titanate (PZT), and is typically often laminated due to a small displacement amount. Other examples of the piezoelectric material include piezoelectric polymers (e.g., polyvinylidene fluoride (PVDF)) and monocrystals (e.g., crystal, LiNbO₃, LiTaO₃, and KNbO₃). The vibration generating unit 20 is preferably disposed per one liquid-column-resonance liquid chamber in a manner that the vibration generating unit 20 can individually control each liquid-column-resonance liquid chamber. It is preferable that the liquid-column-resonance liquid chambers be individually controlled via the elastic plates by partially cutting a block-shaped vibration generating unit, which is formed of one of the above-described materials, according to geometry of the liquid-column-resonance liquid chambers.

As presented in FIG. 2, a plurality of openings are formed in the discharging hole 19. In terms of high productivity, a structure, in which the discharging hole 19 is disposed in the width direction of the liquid-column-resonance liquid chamber 18, is preferably used. Moreover, the frequency of the liquid column resonance is desirably appropriately determined by checking discharging of liquid droplets, because the frequency of the liquid column resonance varies depending on the arrangement of opening of the discharging hole 19.

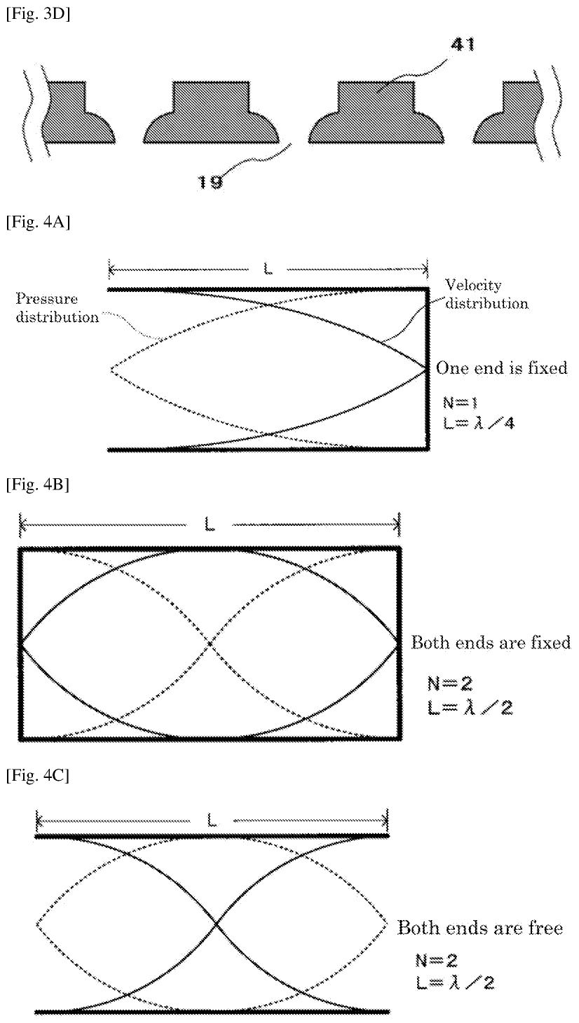

FIGS. 3A to 3D are schematic views presenting exemplary structures of discharging holes. As presented in FIGS. 3A to 3D, cross-sectional shapes of the discharging holes are presented as tapered shapes in which opening diameters gradually decrease from liquid contact surfaces (inlet) towards discharging holes (outlet) of the discharging holes. However, the cross-sectional shapes may be appropriately selected.

As presented in FIG. 3A, the discharging holes 19 have a shape in which an opening diameter gradually decreases from a liquid contact surface towards the discharging hole 19 of the discharging hole while its round shape is maintained. Such a shape can be the most preferable shape from the viewpoint of stable discharging because pressure applied to the liquid at the discharging hole is the largest.

As presented in FIG. 3B, the discharging holes 19 have a shape in which an opening diameter gradually decreases from a liquid contact surface towards a discharging hole 19 of the discharging hole while a certain angle is maintained. Such a shape makes it possible to appropriately change the nozzle angle 24. The shape described in FIG. 3B can increase pressure applied to the liquid adjacent to the discharging holes depending on the nozzle angle, similarly to the shape presented in FIG. 3A.

The nozzle angle 24 is not particularly limited and may be appropriately selected depending on the intended purpose, but is preferably 60 degrees or more but 90 degrees or less. When the nozzle angle is 60 degrees or more, pressure is easily applied to the liquid, resulting in easy processing. When the nozzle angle 24 is 90 degrees or less, pressure is applied adjacent to the outlets of the discharging holes, resulting in stable formation of the liquid droplets. Therefore, the maximum value of the nozzle angle 24 is preferably 90 degrees (corresponding to FIG. 3C).

In FIG. 3D, the discharging holes have a shape obtained by combining the shape presented in FIG. 3A with the shape presented in FIG. 3B. The shape of the discharging holes may be changed stepwise in this way.

A mechanism by which liquid droplets are formed by the liquid droplet formation unit based on the liquid column resonance will now be described.

Firstly, the principle of a liquid column resonance phenomenon that occurs in the liquid-column-resonance liquid chamber 18 of the liquid droplet formation unit 11 presented in FIG. 1 will be described.

A wavelength (k) at which liquid resonance occurs is represented by Expression 1 below:

λ=c/f (Expression 1)

where c denotes sound velocity of the liquid in the liquid-column-resonance liquid chamber; and f denotes a driving frequency applied by the vibration generating unit 20 to the liquid serving as a medium.

In the liquid-column-resonance liquid chamber 18 in FIG. 1, a length from a frame end at a fixed end side to an end at the common liquid supplying path 17 side is represented as L. A height h1 (about 80 μm) of the frame end at the common liquid supplying path 17 side is about 2 times as high as a height h2 (about 40 μm) of a communication hole. The end at the common liquid supplying path side is assumed to be equivalent to a closed fixed end. In such cases where both ends are fixed, resonance is most efficiently formed when the length L corresponds to an even multiple of ¼ of the wavelength (λ). This can be represented by Expression 2 below:

L=(N/4)λ (Expression 2)

In the Expression 2, L denotes a length of the liquid-column-resonance liquid chamber in a longitudinal direction; N denotes an even number; and k denotes a wavelength at which liquid resonance occurs.

The Expression 2 is also satisfied when the both ends are free, that is, the both ends are completely opened.

Likewise, when one end is equivalent to a free end from which pressure is released and the other end is closed (fixed end), that is, when one of the ends is fixed or one of the ends is free, resonance is most efficiently formed when the length L corresponds to an odd multiple of ¼ of the wavelength k. That is, N in the Expression 2 denotes an odd number.

The most efficient driving frequency f is represented by Expression 3 which is derived from the Expression 1 and the Expression 2:

f=N×c/(4L) (Expression 3)

In the Expression 3, L denotes a length of the liquid-column-resonance liquid chamber in a longitudinal direction; c denotes velocity of an acoustic wave of a liquid; and N denotes a natural number.

However, actually, vibration is not amplified unlimitedly because liquid has viscosity which attenuates resonance. Therefore, the resonance has a Q factor, and also occurs at a frequency adjacent to the most efficient driving frequency f calculated according to the Expression 3, as represented by Expression 4 and Expression 5 below.

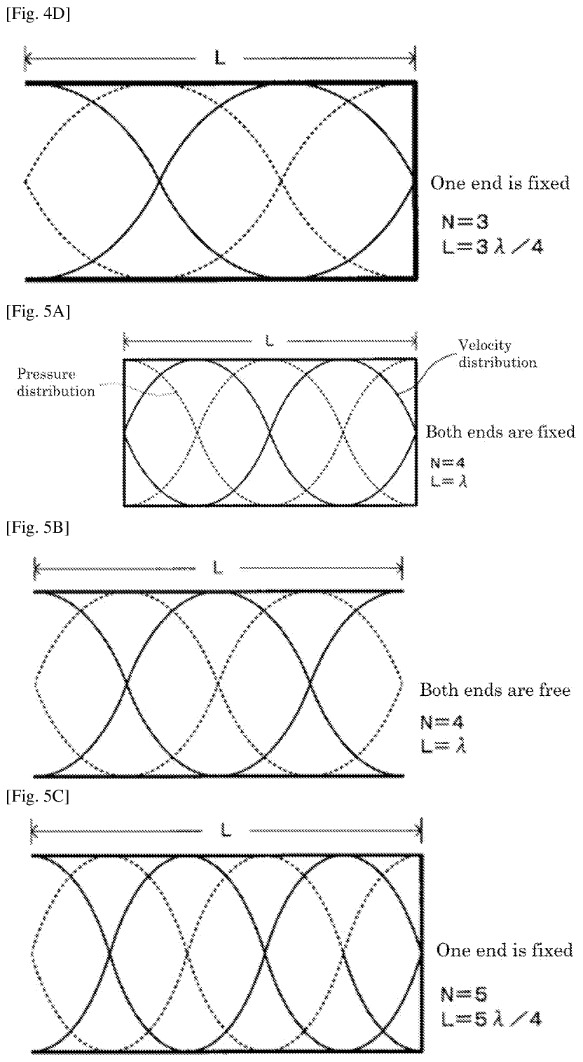

FIG. 4A is a schematic view presenting a standing wave of velocity fluctuation and a standing wave of pressure fluctuation when N=1 and one end is fixed.

FIG. 4B is a schematic view presenting a standing wave of velocity fluctuation and a standing wave of pressure fluctuation when N=2 and both ends are fixed.

FIG. 4C is a schematic view presenting a standing wave of velocity fluctuation and a standing wave of pressure fluctuation when N=2 and both ends are free.

FIG. 4D is a schematic view presenting a standing wave of velocity fluctuation and a standing wave of pressure fluctuation when N=3 and one end is fixed.

FIG. 5A is a schematic view presenting a standing wave of velocity fluctuation and a standing wave of pressure fluctuation when N=4 and both ends are fixed.

FIG. 5B is a schematic view presenting a standing wave of velocity fluctuation and a standing wave of pressure fluctuation when N=4 and both ends are free.

FIG. 5C is a schematic view presenting a standing wave of velocity fluctuation and a standing wave of pressure fluctuation when N=5 and one end is fixed.

In FIGS. 4A to 4D and 5A to 5C, a solid line represents a velocity distribution and a dotted line represents a pressure distribution. Standing wave are actually compressional waves (longitudinal waves), but are commonly expressed as presented in FIGS. 4A to 4D and 5A to 5C. A solid line represents a velocity standing wave and a dotted line represents a pressure standing wave. For example, as can be seen from FIG. 4A in which N=1 and one end is fixed, an amplitude of the velocity distribution is zero at a closed end and the amplitude reaches the maximum at an opened end, which is intuitively understandable. Assuming that a length between both ends of the liquid-column-resonance liquid chamber in a longitudinal direction is L and a wavelength at which liquid column resonance of liquid occurs is k, the standing wave is most efficiently generated when the integer N is from 1 through 5. A standing wave pattern varies depending on whether each end is opened or closed. Therefore, standing wave patterns under various opening/closing conditions are also described in the drawings. As described below, conditions of the ends are determined depending on states of openings of the discharging holes and states of openings at a supplying side.

Note that, in the acoustics, an opened end refers to an end at which moving velocity of a medium reaches the local maximum, but, to the contrary, pressure of the medium is zero. Conversely, a closed end refers to an end at which moving velocity of a medium (liquid) is zero in a longitudinal direction, but, to the contrary, pressure of the medium reaches the local maximum. The closed end is considered as an acoustically hard wall and reflects a wave. When an end is ideally perfectly closed or opened, resonance standing waves as presented in FIGS. 4A to 4D and 5A to 5C are formed by superposition of waves. However, standing wave patterns vary depending on the number of the discharging holes and positions at which the discharging holes are opened. Therefore, a resonance frequency appears at a position shifted from a position determined according to the Expression 3. In this case, conditions under which liquid droplets are stably formed can be created by appropriately adjusting the driving frequency. For example, when the sound velocity c of the liquid is 1,200 m/s, the length L of the liquid-column-resonance liquid chamber is 1.85 mm, and a resonance mode, in which both ends are completely equivalent to fixed ends due to the presence of walls on the both ends and N=2, is used, the most efficient resonance frequency is calculated as 324 kHz from the Expression 2. In another example, when the sound velocity c of the liquid is 1,200 m/s and the length L of the liquid-column-resonance liquid chamber is 1.85 mm, these conditions are the same as above, and a resonance mode, in which both ends are equivalent to fixed ends due to the presence of walls at the both ends and N=4, is used, the most efficient resonance frequency is calculated as 648 kHz from the Expression 2. Thus, a higher-order resonance can be utilized even in a liquid-column-resonance liquid chamber having the same configuration.

In order to increase the frequency, the liquid-column-resonance liquid chamber of the liquid droplet formation unit 11 presented in FIG. 1 preferably has both ends which are equivalent to a closed end or are considered as an acoustically soft wall due to influence from openings of the discharging holes. However, the both ends may be free. The influence from openings of the discharging holes means decreased acoustic impedance and, in particular, an increased compliance component. Therefore, the configuration, in which walls are formed at both ends of the liquid-column-resonance liquid chamber in a longitudinal direction, as presented in FIGS. 4B and 5A, is preferable because it is possible to use both of a resonance mode in which both ends are fixed and a resonance mode in which one of ends is free, that is, an end at a discharging hole side is considered to be opened.

The number of openings of the discharging holes, positions at which the openings are disposed, and cross-sectional shapes of the discharging holes are also factors which determine the driving frequency. The driving frequency can be appropriately determined based on these factors. For example, when the number of the discharging holes is increased, the liquid-column-resonance liquid chamber gradually becomes free at an end which has been fixed. As a result, a resonance standing wave which is approximately the same as a standing wave at the opened end is generated and the driving frequency is increased. Further, the end which has been fixed becomes free starting from a position at which an opening of the discharging hole that is the closest to the liquid supplying path is disposed. As a result, a cross-sectional shape of the discharging hole is changed to a round shape or a volume of the discharging hole is varied depending on a thickness of the frame, so that an actual standing wave has a shorter wavelength and a higher frequency than the driving frequency. When a voltage is applied to the vibration generating unit at the driving frequency determined as described above, the vibration generating unit deforms and the resonance standing wave is generated most efficiently at the driving frequency. The liquid column resonance standing wave is also generated at a frequency adjacent to the driving frequency at which the resonance standing wave is generated most efficiently. That is, assuming that a length between both ends of the liquid-column-resonance liquid chamber in a longitudinal direction is L and a distance to a discharging hole that is the closest to an end at a liquid supplying side is Le, the driving frequency f is determined according to Expression 4 and Expression 5 below using both of the lengths L and Le. A driving waveform having, as a main component, the driving frequency f can be used to vibrate the vibration generating unit and to induce the liquid column resonance to thereby discharge the liquid droplets from the discharging holes for formation of liquid droplets.

N×c/(4L)≤f≤N×c/(4Le) (Expression 4)

N×c/(4L)≤f≤(N+1)×c/(4Le) (Expression 5)

In the Expressions 4 and 5, L denotes a length of the liquid-column-resonance liquid chamber in a longitudinal direction; Le denotes a distance from an end at a liquid supplying path side to a center of a discharging hole that is the closest to the end; c denotes velocity of an acoustic wave of a liquid; and N denotes a natural number.

Note that, a ratio (L/Le) of the length L between both ends of the liquid-column-resonance liquid chamber in a longitudinal direction to the distance Le to the discharging hole that is the closest to the end at the liquid supplying side preferably satisfies Expression 6 below.

Le/L>0.6 (Expression 6)

Based on the principle of the liquid column resonance phenomenon described above, a liquid-column resonance pressure standing-wave is formed in the liquid-column-resonance liquid chamber 18 presented in FIG. 1, and continuous discharging is performed from the discharging holes 19 disposed in a portion of the liquid-column-resonance liquid chamber 18, to thereby form liquid droplets. Note that, the discharging hole 19 is preferably disposed at a position at which pressure of the standing wave varies to the greatest extent from the viewpoints of high efficiency of forming liquid droplets and driving at a lower voltage. One liquid-column-resonance liquid chamber 18 may include one discharging hole 19, but preferably includes two or more (a plurality of) discharging holes from the viewpoint of productivity. Specifically, the number of discharging holes is preferably 2 or more but 100 or less. When the number of discharging holes is 2 or more, productivity can be improved. When the number of discharging holes is 100 or less, a voltage to be applied to the vibration generating unit 20 may be set at a low level in order to form desired liquid droplets from the discharging holes 19. As a result, a piezoelectric material can stably behave as the vibration generating unit 20.

When the plurality of the discharging holes 19 are disposed, a pitch between the discharging holes (the shortest distance between centers of discharging holes adjacent to each other) is preferably 20 μm or longer but equal to or shorter than the length of the liquid-column-resonance liquid chamber. When the pitch between the discharging holes is 20 μm or more, it is possible to decrease the possibility that liquid droplets, which are discharged from discharging holes adjacent to each other, collide with each other to form a larger droplet. As a result, particles having a good particle diameter distribution may be obtained.

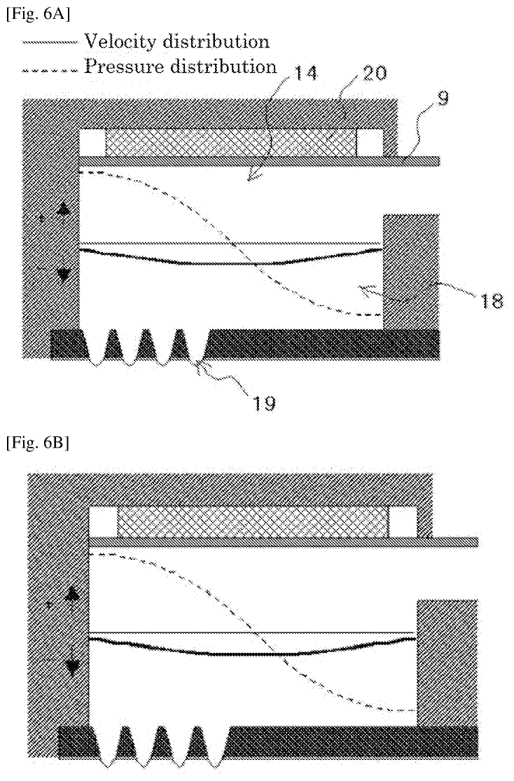

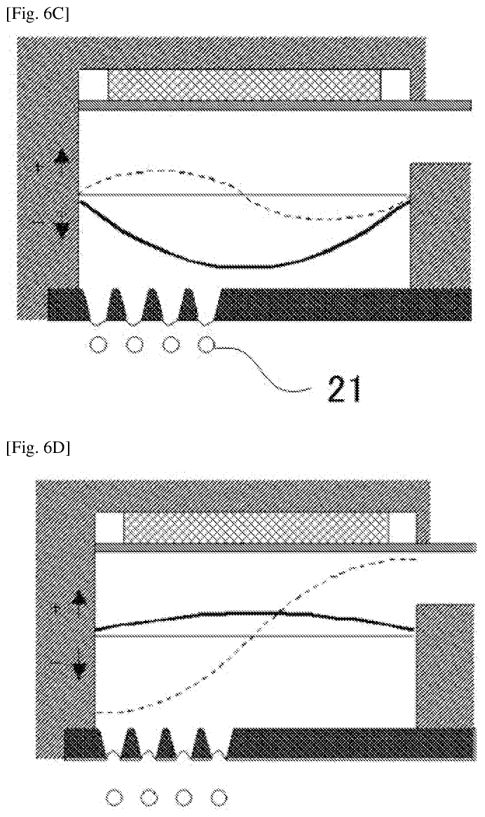

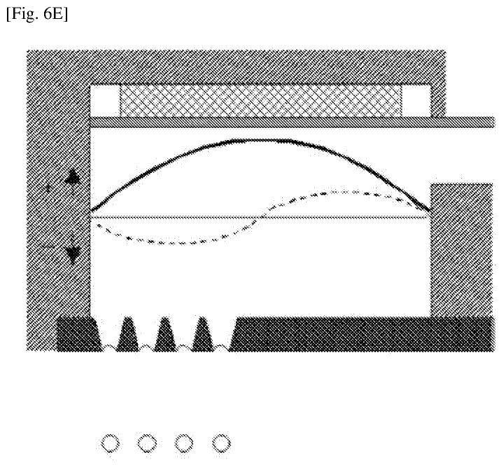

Next, a liquid column resonance phenomenon which occurs in the liquid-column-resonance liquid chamber of a liquid-droplet discharging head of the liquid droplet formation unit will be described with reference to FIGS. 6A to 6E. Note that, in FIGS. 6A to 6E, a solid line drawn in the liquid-column-resonance liquid chamber represents a velocity distribution plotting velocity at arbitrary measuring positions between an end at the fixed end side and an end at the common liquid supplying path side in the liquid-column-resonance liquid chamber. A direction from the common liquid supplying path to the liquid-column-resonance liquid chamber is assumed as plus (+), and the opposite direction is assumed as minus (−). A dotted line drawn in the liquid-column-resonance liquid chamber represents a pressure distribution plotting pressure at arbitrary measuring positions between an end at the fixed end side and an end at the common liquid supplying path side in the liquid-column-resonance liquid chamber. A positive pressure relative to atmospheric pressure is assumed as plus (+), and a negative pressure is assumed as minus (−). In the case of the positive pressure, pressure is applied in a downward direction in the drawings. In the case of the negative pressure, pressure is applied in an upward direction in the drawings. In FIGS. 6A to 6E, as described above, the end at the liquid supplying path side is free, and the height of the frame serving as the fixed end (height h1 in FIG. 1) is about 2 times or more as high as the height of an opening at which the liquid supplying path is in communication with the liquid-column-resonance liquid chamber 18 (height h2 in FIG. 1). Therefore, FIGS. 6A to 6E represent temporal changes of a velocity distribution and a pressure distribution under an approximate condition in which the liquid-column-resonance liquid chamber 18 are approximately fixed at both ends. In FIGS. 6A to 6E, a solid line represents a velocity distribution and a dotted line represents a pressure distribution.

A schematic view presenting one example of liquid column resonance phenomenon that occurs in a liquid column resonance flow path of a liquid droplet formation unit.

FIG. 6A presents a pressure waveform and a velocity waveform in the liquid-column-resonance liquid chamber 18 at a time when liquid droplets are discharged. In FIG. 6B, meniscus pressure is increased again after the liquid droplets are discharged and immediately then the liquid is drawn. As presented in FIGS. 6A and 6B, pressure in a flow path, on which the discharging holes 19 are disposed, in the liquid-column-resonance liquid chamber 18 is the local maximum. Then, as presented in FIG. 6C, positive pressure adjacent to the discharging holes 19 is decreased and shifted to a negative pressure side. Thus, the liquid droplets 21 are discharged.

Then, as presented in FIG. 6D, the pressure adjacent to the discharging holes 19 is the local minimum. From this time point, the liquid-column-resonance liquid chamber 18 starts to be filled with the liquid 14. Then, as presented in FIG. 6E, negative pressure adjacent to the discharging holes 19 is decreased and shifted to a positive pressure side. At this time point, the liquid chamber is completely filled with the liquid 14. Then, as presented in FIG. 6A, positive pressure in a liquid-droplet discharging region of the liquid-column-resonance liquid chamber 18 is the local maximum again to discharge the liquid droplets 21 from the discharging holes 19. Thus, the liquid column resonance standing wave is generated in the liquid-column-resonance liquid chamber by the vibration generating unit driven at a high frequency. The discharging holes 19 are disposed in the liquid-droplet discharging region corresponding to the anti-nodes of the liquid column resonance standing wave at which pressure varies to the greatest extent. Therefore, the liquid droplets 21 are continuously discharged from the discharging holes 19 in synchronized with an appearance cycle of the anti-nodes.

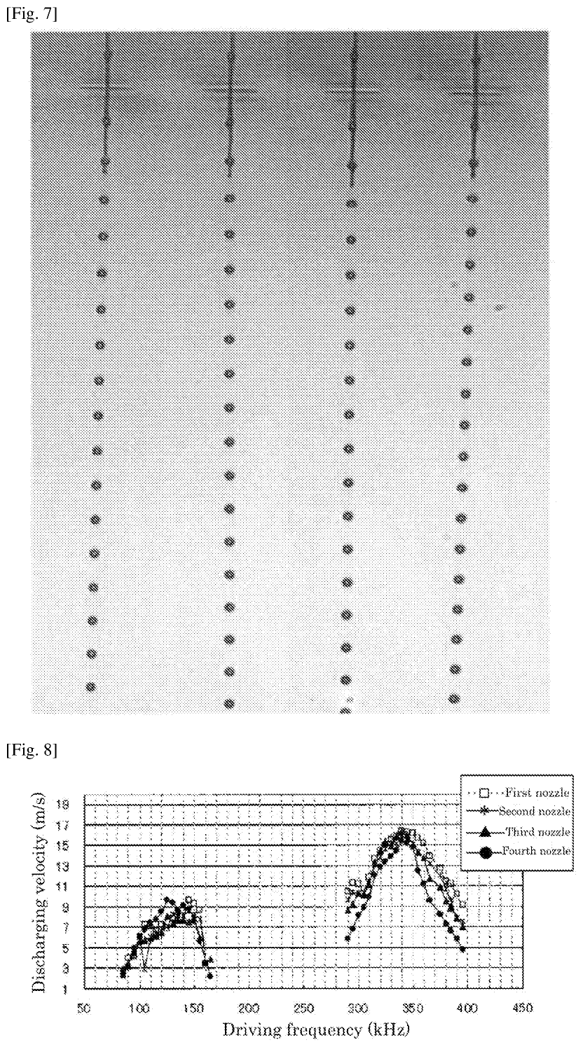

One exemplary aspect where liquid droplets are actually discharged based on the liquid column resonance phenomenon will now be described. FIG. 7 is an image presenting exemplary actual liquid droplets discharged by a liquid droplet formation unit. In this example, liquid droplets were discharged under the below-described conditions: the length L between both ends of the liquid-column-resonance liquid chamber 18 in a longitudinal direction in FIG. 1 was 1.85 mm, a resonance mode was N=2, the first to fourth discharging holes were disposed at positions corresponding to anti-nodes of a pressure standing wave in the resonance mode of N=2, and the driving frequency was a sine wave at 340 kHz. FIG. 7 is a photograph of the thus-discharged liquid droplets, and the photograph was taken by laser shadowgraphy. As can be seen from FIG. 7, the liquid droplets which are very uniform in diameter and substantially uniform in velocity are successfully discharged.

FIG. 8 is a graph presenting dependency of a liquid droplet-discharging velocity on a driving frequency when driven by a sine wave having the same amplitude of 290 kHz or more but 395 kHz or less as the driving frequency. As can be seen from FIG. 8, a discharging velocity of liquid droplets from each of the first to fourth nozzles is uniform and is the maximum discharging velocity adjacent to the driving frequency of about 340 kHz. It can be seen from this result that uniform discharging is achieved at a position corresponding to an anti-node of the liquid column resonance standing wave at 340 kHz which is the second mode of a liquid column resonance frequency. It can also be seed from the results in FIG. 8 that a frequency characteristic of liquid column resonance standing waves characteristic of the liquid column resonance occurs in the liquid-column-resonance liquid chamber. The frequency characteristic is that liquid droplets are not discharged between a liquid droplet-discharging velocity peak at 130 kHz, which is the first mode, and a liquid droplet-discharging velocity peak at 340 kHz, which is the second mode.

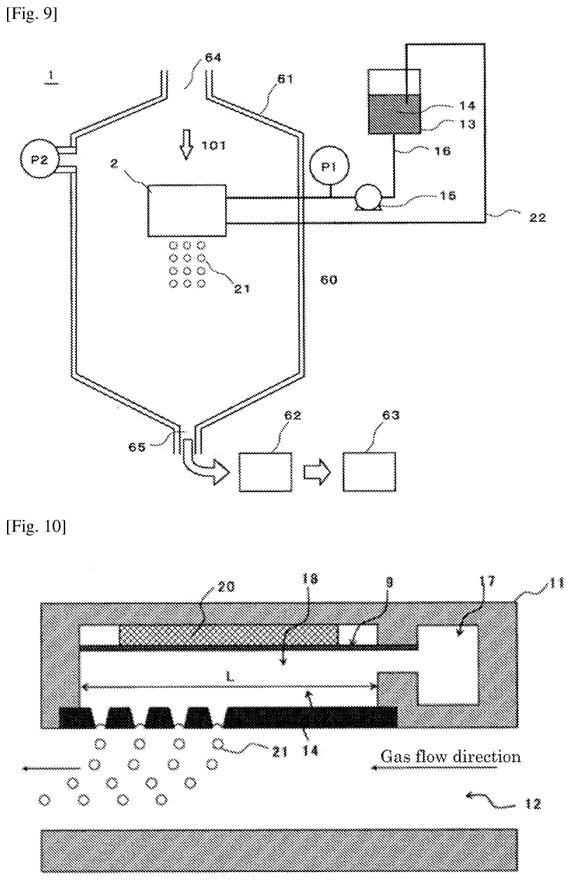

FIG. 9 is a schematic view presenting one example of a particle production apparatus. A particle production apparatus 1 presented in FIG. 9 mainly includes a liquid droplet formation unit 2, a drying collection unit 60, a conveyance gas flow discharging port 65, and a particle storage section 63. The liquid droplet formation unit 2 is coupled to a raw material housing container 13 configured to house a liquid 14 through a liquid supplying pipe 16 and a liquid returning pipe 22. The liquid supplying pipe 16 is coupled to a liquid circulating pump 15. The liquid circulating pump 15 is configured to supply the liquid 14 housed in the raw material housing container 13 to the liquid droplet formation unit 2 through the liquid supplying pipe 16, and to feed the liquid 14 in the liquid supplying pipe 16 under pressure to return to the raw material housing container 13 through a liquid returning pipe 22. This configuration makes it possible to supply the liquid 14 to the liquid droplet formation unit 2 at all times. The liquid supplying pipe 16 is provided with a pressure gauge P1 and the drying ⋅ collection unit 60 is provided with a pressure gauge P2. The pressure at which the liquid is fed to the liquid droplet formation unit 2 and the pressure within the drying ⋅ collection unit are controlled by pressure gauges P1 and P2. When a value of pressure measured at P1 is larger than a value of pressure measured at P2, there is a risk that the liquid 14 is oozed from the discharging hole. When a value of pressure measured at P1 is smaller than a value of pressure measured at P2, there is a risk that a gas enters the liquid droplet formation unit 2 to stop discharging. Therefore, it is preferable that a value of pressure measured at P1 and a value of pressure measured at P2 be substantially the same.

Within a chamber 61, a downward gas flow (conveyance gas flow) 101 generated from a conveyance gas flow introducing port 64 is formed. A liquid droplet 21 discharged from the liquid droplet formation unit 2 is conveyed downward not only through gravity but also through the conveyance gas flow 101, passes through the conveyance gas flow discharging port 65, is collected by a collecting unit 62, and is stored in the particle storage section 63.

When discharged liquid droplets contact with each other before they are dried, the liquid droplets are unified to form a single particle (hereinafter, this phenomenon may be referred to as “cohesion”). In order to obtain a particle having a uniform particle size distribution, it is necessary to maintain a distance between the discharged liquid droplets. Although the discharged liquid droplet travels at a certain initial velocity, the velocity is decreased soon due to air resistance. The liquid droplet decreased in the velocity is caught up with by a liquid droplet subsequently discharged, which leads to cohesion. This phenomenon occurs regularly. Therefore, when a particle is collected, the particle size distribution considerably becomes worsened. In order to prevent cohesion, it is preferable to dry (solidify) and convey liquid droplets, while the velocity of the liquid droplet is prevented from being decreased and the liquid droplets do not contact with each other to prevent cohesion by the conveyance gas flow 101. Finally, it is preferable to convey the particle to the collecting unit.

As presented in FIG. 9, a part of the conveyance gas flow 101 as the first gas flow is provided near the liquid droplet formation unit in the same direction as the direction in which the liquid droplet is discharged. As a result, the velocity of the liquid droplet immediately after the liquid droplet is discharged is prevented from being decreased, which makes it possible to prevent cohesion. FIG. 10 is a schematic view presenting one exemplary gas flow path. The gas flow in the gas flow path 12 may be orientated in a direction transverse to the liquid-droplet discharging direction, as presented in FIG. 10. Alternatively, although not presented, the gas flow may be oriented at a certain angle, and the certain angle is preferably such an angle that the liquid droplets are spaced from each other by the liquid droplet formation unit. As presented in FIG. 10, when a cohesion preventing gas flow is provided from the direction transverse to the direction in which the liquid droplet is discharged, the cohesion preventing gas flow is preferably orientated in a direction in which trajectories of the liquid droplets do not overlap with each other when the liquid droplets are conveyed from the discharging holes by the cohesion preventing gas flow.

After cohesion is prevented by the first gas flow as described above, the dried particles may be conveyed to the collecting unit by the second gas flow.

The velocity of the first gas flow is preferably equal to or higher than the velocity of the liquid droplet to be discharged. When the velocity of the cohesion preventing gas flow is lower than the velocity of the liquid droplet to be discharged, it may be difficult to exhibit a function of preventing liquid droplets from contacting with each other, which is an original purpose of the cohesion preventing gas flow.

As a property of the first gas flow, such a condition that the liquid droplets do not cohere can be added, and the property of the first gas flow may be different from that of the second gas flow. Moreover, such a chemical substance that facilitates drying of the surface of the particle may be mixed with or added to the cohesion preventing gas flow, in expectation of physical action.

A state of the conveyance gas flow 101 is not particularly limited to a state of the gas flow. The conveyance gas flow 101 may be a laminar flow, a rotational flow, or a turbulent flow. Kinds of gases constituting the conveyance gas flow 101 are not particularly limited and may be appropriately selected depending on the intended purpose. For example, air may be used, or an incombustible gas such as nitrogen may be used. A temperature of the conveyance gas flow 101 can be appropriately adjusted. Preferably, the temperature thereof is not changed at the time of production. A unit configured to change a gas flow condition of the conveyance gas flow 101 may be included within the chamber 61. The conveyance gas flow 101 may be used not only for prevention of cohesion of the liquid droplets 21 but also for prevention of attachment to the chamber 61.

When an amount of the residual solvent contained in the particle obtained by the particle collecting unit presented in FIG. 9 is large, the secondary drying is preferably performed if necessary in order to decrease the residual solvent. As the secondary drying, generally known drying units such as fluidized bed drying and vacuum drying can be used.

### EXAMPLES

Examples of the present disclosure will be described hereinafter. However, the present disclosure should not be construed as being limited to these Examples.

### Example 1

<Preparation of Liquid A>

Tyrphostin (obtained from Tokyo Chemical Industry Co., Ltd.) (2 parts by mass) and lactose monohydrate (obtained from Tokyo Chemical Industry Co., Ltd.) (8 parts by mass) were added to a mixture solvent of water (700 parts by mass) and methanol (300 parts by mass), followed by dissolving the resultant to obtain liquid A.

<Production of Instantly Soluble Particle 1>

A liquid column resonance droplet-discharging apparatus (obtained from Ricoh Company, Ltd.) of FIG. 1, in which the number of openings of the discharging holes was set to one per one liquid-column-resonance liquid chamber, was used to discharge the obtained liquid A from the discharging hole to form liquid droplets. Then, the apparatus of FIG. 9 was used to dry the liquid droplets to obtain instantly soluble particle 1. Here, particle formulation conditions were as follows.

—Particle Formulation Conditions—

——Liquid Column Resonance Conditions——
Resonance mode (N): 2
Length (L) between both ends in longitudinal direction of liquid-column-resonance liquid chamber: 1.8 mm
Height (h1) of end of frame at common liquid supplying path side of liquid-column-resonance liquid chamber: 80 μcm
Height (h2) of communication hole of liquid-column-resonance liquid chamber: 40 μcm

——Liquid Droplet Formation Unit——
Shape of discharging hole: perfect circle
Diameter of discharging hole: 8.0 μm
Number of openings of discharging holes: 1 (per one liquid-column-resonance liquid chamber)
Number of liquid-column-resonance liquid chambers: 384 chambers
Applied voltage: 12.0 V
Driving frequency: 310 kHz

——Particle Formulation Unit——
Conveyance gas flow: dry nitrogen
Temperature of conveyance gas flow: 40 degrees Celsius
Flow rate of conveyance gas flow: 100 L/min

### Example 2

<Preparation of Liquid B>

Liquid B was prepared in the same manner as in Example 1 except that the formulation in Example 1 was changed to the formulation presented in Table 1.

<Production of Instantly Soluble Particle 2>

Instantly soluble particle 2 was obtained in the same manner as in Example 1 except that a spray dryer (apparatus name: GS310, obtained from Yamato Scientific Co., Ltd.) was used to form the obtained liquid B into liquid droplets. Here, particle formulation conditions were as follows.

—Particle Formulation Conditions—

——Liquid Droplet Formation Unit——
Shape of discharging hole: perfect circle
Diameter of discharging hole: 0.5 mm
Number of openings of discharging holes: 1
Discharging air pressure: 0.1 MPa

——Particle Formulation Unit——
Conveyance gas flow: dry nitrogen
Temperature of conveyance gas flow: 75 degrees Celsius
Flow rate of conveyance gas flow: 500 L/min

(Examples 3 to 6 and 9 to 11) Liquids C to F and I to K were prepared and instantly soluble particles 3 to 6 and 9 to 11 were obtained in the same manner as in Example 1 except that the formulation in Example 1 was changed to the formulation presented in Table 1.

### Example 7

<Preparation of Liquid G>

Liquid G was prepared in the same manner as in Example 1 except that the formulation in Example 1 was changed to the formulation presented in Table 1.

<Production of Instantly Soluble Particle 7>

Instantly soluble particle 7 was obtained in the same manner as in Example 3 except that a particle production apparatus provided with a film vibration-type nozzle (obtained from Optnics Precision CO., LTD.) was used. Here, particle formulation conditions were as follows.

—Particle Formulation Conditions—

——Liquid Droplet Formation Unit——
Shape of discharging hole: perfect circle
Diameter of discharging hole: 10 μm
Applied voltage: 20.0 V
Driving frequency: 100 KHz

——Particle Formulation Unit——
Temperature of dry air: 40 degrees Celsius
Flow rate of dry air: 500 L/min

### Example 8

Liquid H was prepared and instantly soluble particle 8 was obtained in the same manner as in Example 2 except that the formulation in Example 2 was changed to the formulation presented in Table 1.

### Comparative Examples 1 and 2

Liquids L and M were prepared and particles 12 and 13 were obtained in the same manner as in Example 1 except that the formulation in Example 1 was changed to the formulation presented in Table 1; and tyrphostin as the poorly water-soluble compound, a water-soluble polymer (HPC-L and Eudragit RL), which is not a rapidly water-soluble compound, as the water-soluble base material, water as the solvent 1, and methanol or acetone as the solvent 2 were used.

TABLE 1

| | | | Poorly-water-soluble compound | | | Water-soluble | | | | | |
|---|---|---|---|---|---|---|---|---|---|---|---|
| | | | (pharmaceutical compound) | | | base material | | Solvent 1 | | Solvent 2 | |
| | | | | Amount | | | Amount | | Amount | | Amount |
| | | | | (part [symbol] by | Solubility | | (parts by | | (parts by | | (parts by |
| | | Liquid | Name | mass) | (μg/mL) | Name | mass) | Name | mass) | Name | mass) |
| Ex. | 1 | A | Tryphostin | 2 | 0.6 | Lactose | 8 | Water | 700 | Methanol | 300 |
| | | | | | | monohydrate | | | | | |
| | 2 | B | Tryphostin | 2 | 0.6 | Lactose | 8 | Water | 350 | Methanol | 150 |
| | | | | | | monohydrate | | | | | |
| | 3 | C | Tryphostin | 7 | 0.6 | Lactose | 3 | Water | 700 | Methanol | 300 |
| | | | | | | monohydrate | | | | | |
| | 4 | D | Tryphostin | 2 | 0.6 | Mannitol | 8 | Water | [symbol] | Methanol | [symbol] |
| | 5 | E | [symbol] nib | 5 | 20 | Glucose | 5 | Water | 700 | Ethanol | 300 |
| | 6 | F | [symbol] nib | 7 | 20 | Lactose | 5 | Water | 700 | Ethanol | 300 |
| | | | | | | monohydrate | | | | | |
| | 7 | G | [symbol] nib | 2 | 20 | Mannitol | 8 | Water | 700 | Ethanol | 300 |
| | 8 | H | [symbol] | 2 | 20 | Glucose | 8 | Water | [symbol] | Ethanol | 100 |
| | 9 | I | [symbol] | [symbol] | [symbol] | Lactose | 18 | Water | 700 | [symbol] | 300 |
| | | | hydrochloride | | | monohydrate | | | | | |
| | 10 | J | [symbol] | 1 | 15 | Lactose | [symbol] | Water | 700 | Ethanol | 300 |
| | | | | | | monohydrate | | | | | |
| | 11 | K | [symbol] profen | [symbol] | 20 | Lactose | 18 | Water | 700 | Ethanol | 300 |
| | | | | | | monohydrate | | | | | |
| Comp. | 1 | L | Tryphostin | 2 | 0.6 | HPC-L | 8 | Water | 230 | Methanol | 100 |
| Ex. | 2 | M | Tryphostin | 2 | 0.6 | Endragit RL | 8 | Water | 500 | [symbol] | [symbol] |

[symbol]

indicates data missing or illegible when filed

Particles 1 to 13 obtained in Examples 1 to 11 and Comparative Examples 1 and 2 were measured and evaluated for “amount of poorly water-soluble compound in particle”, “volume average particle diameter”, “instant solubility”, and “X-ray diffraction” in the following manners. Results are presented in Table 2.

<Amount of Poorly Water-Soluble Compound in Particle>

An amount of the poorly water-soluble compound in each of the produced particles 1 to 13 was quantified through liquid chromatograph (detector: UV/Vis detector). Results are presented in Table 2.

<Volume Average Particle Diameter>

The volume average particle diameter was measured using a laser diffraction/scattering particle size distribution analyzer (device name: MICROTRAC MT3000II, obtained from MicrotracBEL Corp.).

<Evaluation of Instant Solubility>

The formed particle was weighed in an amount twice the mass thereof that reached the saturation solubility of an active pharmaceutical ingredient (poorly water-soluble compound), and was added to a physiological saline solution (10 mL) After the addition, a dissolution state when the resultant was shaken by hand at two times/sec was evaluated based on the following evaluation criteria. The time of the handshake was performed with three levels (10 seconds, 20 seconds, and 30 seconds), and the dissolution state at that time was confirmed. Considering practical use in clinical sites, A, B, and C were considered acceptable. Results are presented in Table 2. Note that, the phrase “the particle was completely dissolved” means that it can be visually confirmed that there is no remaining particle.

—Evaluation Criteria—

A:

Completely dissolved within 10 seconds.

B: Completely dissolved within 20 seconds.

C: Completely dissolved within 30 seconds.

D: Longer than 30 seconds.

<Measurement of X-Ray Diffraction>

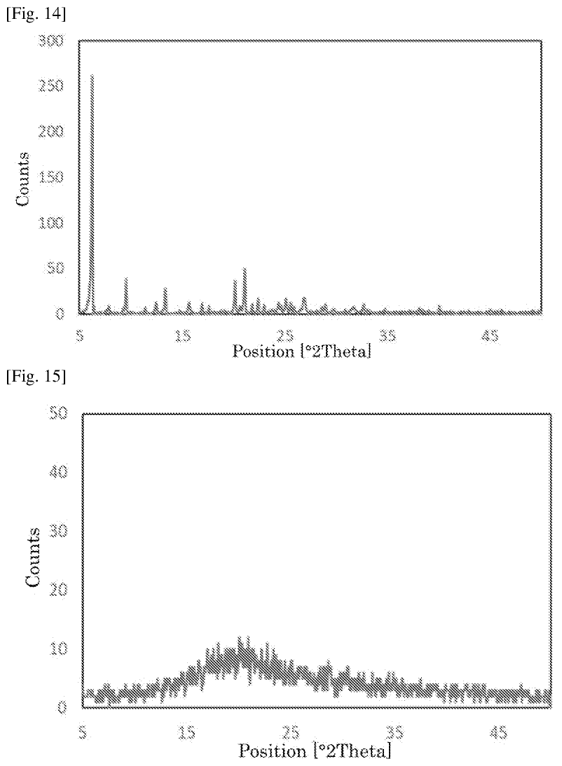

The produced particle was evaluated for presence or absence of crystallinity using an X-ray diffractometer (obtained from X'Pert PHILIPS). Results of X-ray diffraction were evaluated as follows. Specifically, when the result of X-ray diffraction was a halo pattern, the particle was evaluated to be in an amorphous state. When a defined peak derived from the pharmaceutical agent was found, the particle was evaluated to be in a crystalline state. Results are presented in Table 2. FIG. 14 presents a diffraction pattern of the poorly water-soluble compound (erlotinib) alone used in Example 9, which was obtained through measurement of the X-ray diffraction. FIG. 15 presents a diffraction pattern of the particle 9 obtained in Example 9, which was obtained through measurement of the X-ray diffraction.

TABLE 2

| | | | | Evaluation criteria | | | |
|---|---|---|---|---|---|---|---|
| | | | | Amount of poorly- | Volume | | |
| | | | | water- soluble | average | | |
| | | | | compound in | particle | | Measurement |
| | | Particle | Particle formation | particle | diameter | Instant | of X-ray |
| | | No. | conditions | (% by mass) | (μm) | solubility | diffraction |
| Ex. | 1 | 1 | Liquid column | 20 | 2.5 | A | Amorphous |
| | | | resonance | | | | |
| | 2 | 2 | Two-fluid spray | 20 | 4.8 | B | Amorphous |
| | 3 | 3 | Liquid column | 70 | 2.4 | C | Amorphous |
| | | | resonance | | | | |
| | 4 | 4 | Liquid column | 20 | 3.1 | A | Amorphous |
| | | | resonance | | | | |
| | 5 | 5 | Liquid column | 50 | 2.5 | B | Amorphous |
| | | | resonance | | | | |
| | 6 | 6 | Liquid column | 70 | 2.5 | C | Amorphous |
| | | | resonance | | | | |
| | 7 | 7 | Film vibration | 20 | 5.2 | B | Amorphous |
| | 8 | 8 | Two-fluid spray | 20 | 3.2 | B | Amorphous |
| | 9 | 9 | Liquid column | 10 | 2.6 | B | Amorphous |
| | | | resonance | | | | |
| | 10 | 10 | Liquid column | 10 | 2.5 | B | Amorphous |
| | | | resonance | | | | |
| | 11 | 11 | Liquid column | 10 | 2.5 | A | Amorphous |
| | | | resonance | | | | |
| Comp. | 1 | 12 | Liquid column | 20 | 4.2 | D | Amorphous |
| Ex. | | | resonance | | | | |
| | 2 | 13 | Liquid column | 20 | 2.5 | D | Amorphous |
| | | | resonance | | | | |

In Examples 1 to 13, all of the powdery pharmaceutical preparations were completely dissolved within 30 seconds to obtain uniformly transparent solutions. Meanwhile, in Comparative Examples 1 and 2, the whole aqueous solutions were found to have cloudiness even after 30 seconds.

### Test Example 1

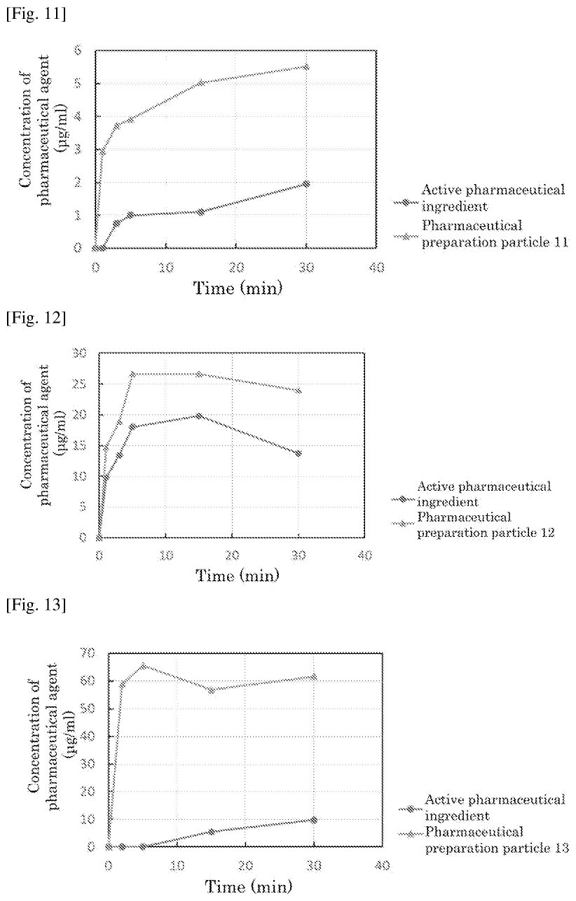

An active pharmaceutical ingredient of erlotinib (1.6 mg) and the particle 9 (16 mg) obtained in Example 9 were each added to a physiological saline solution (0.9 w/v %) (100 mL) as a solvent, and were dissolved for 30 minutes under the following conditions: a temperature of the solution: 25 degrees Celsius; and a stirring rate: 50 rpm. Then, an amount of the pharmaceutical agent dissolved over time was quantified through liquid chromatography (detector: UV/Vis detector). Results are presented in Table 3 and FIG. 11.

### Test Example 2

An active pharmaceutical ingredient of imatinib (7.35 mg) and the particle 10 (73.5 mg) obtained in Example 10 were each added to water (100 mL), and were dissolved for 30 minutes under the following conditions: a temperature of the solution: 25 degrees Celsius; and a stirring rate: 50 rpm. Then, an amount of the pharmaceutical agent dissolved over time was quantified through liquid chromatography (detector: UV/Vis detector). Results are presented in Table 4 and FIG. 12.

### Test Example 3

An active pharmaceutical ingredient of ibuprofen (10.5 mg) and the particle 11 (105 mg) obtained in Example 11 were each added to a physiological saline solution (0.9 wt %) (100 mL) as a solvent, and were dissolved for 30 minutes under the following conditions: a temperature of the solution: 25 degrees; and a stirring rate: 50 rpm. Then, an amount of the pharmaceutical agent dissolved over time was quantified through liquid chromatography (detector: UV/Vis detector). Results are presented in Table 5 and FIG. 13.

TABLE 3

| | | Test Example 1 | |
|---|---|---|---|
| | | Dissolution amount (mg/mL) | |
| | | Erlotinib | Erlotinib |
| | | active | pharmaceutical |
| | | pharmaceutical | preparation |
| | | ingredient | particle 9 |
| Time | 1 | Not detected | 2.9 |
| (min) | 3 | 0.8 | 3.7 |
| | 5 | 1 | 3.9 |
| | 15 | 1.1 | 5 |
| | 30 | 2 | 5.5 |

TABLE 4

| | | Test Example 2 | |
|---|---|---|---|
| | | Dissolution amount (mg/mL) | |
| | | Imatinib | Imatinib |
| | | active | pharmaceutical |
| | | pharmaceutical | preparation |
| | | ingredient | particle 10 |
| Time | 1 | 9.7 | 14.7 |
| (min) | 3 | 13.4 | 18.9 |
| | 5 | 18 | 26.6 |
| | 15 | 19.8 | 26.6 |
| | 30 | 13.7 | 24 |

TABLE 5

| | | Test Example 3 | |
|---|---|---|---|
| | | Dissolution amount (mg/mL) | |
| | | Ibuprofen | Ibuprofen |
| | | active | pharmaceutical |
| | | pharmaceutical | preparation |
| | | ingredient | particle 11 |
| Time | 2 | Not detected | 58.9 |
| (min) | 5 | Not detected | 65.6 |
| | 15 | 5.4 | 56.8 |
| | 30 | 9.7 | 61.6 |

As presented in Tables 3 to 5, in all of the Test Examples 1 to 3, the pharmaceutical preparation particles prepared in Examples exhibited a higher concentration of the pharmaceutical preparation than the active pharmaceutical ingredients, in an early stage of dissolution. Even after thirty minutes from the start of the dissolution, the particles of Examples exhibited a considerably higher concentration of the pharmaceutical agent than the active pharmaceutical ingredients. It is presumed that the reason for this is because the poorly water-soluble compound exists in a rapidly water-soluble base material in an amorphous state to thereby improve the instant solubility and the solubility of the pharmaceutical agent.

Aspects of the present disclosure are as follows, for example.

<1> An instantly soluble particle including:

a water-soluble base material; and

a poorly water-soluble compound,

wherein the water-soluble base material contains a rapidly water-soluble compound, and

the poorly water-soluble compound exists in the water-soluble base material in an amorphous state.

<2> The instantly soluble particle according to <1>, wherein the rapidly water-soluble compound is at least one selected from the group consisting of monosaccharides and disaccharides.

<3> The instantly soluble particle according to <1> or <2>, wherein an amount of the poorly water-soluble compound is 75% by mass or less.

<4> The instantly soluble particle according to any one of <1> to <3>, wherein the amount of the poorly water-soluble compound is 10% by mass or more but 50% by mass or less.

<5> The instantly soluble particle according to any one of <1> to <4>, wherein a volume average particle diameter (Dv) of the instantly soluble particle is 0.5 μm or more but 50 μm or less.

<6> The instantly soluble particle according to any one of <1> to <5>, wherein the volume average particle diameter (Dv) of the instantly soluble particle is 0.5 μm or more but 20 μm or less.

<7> A method for producing an instantly soluble particle, the method including: discharging a liquid containing a rapidly water-soluble compound and a poorly water-soluble compound from a discharging hole to form liquid droplets; and solidifying the liquid droplets.

<8> The method for producing an instantly soluble particle according to <7>, wherein the discharging includes applying vibration to a liquid composition housed in a liquid-column-resonance liquid chamber to form standing waves through liquid column resonance and discharging the liquid composition from the discharging hole, the discharging hole being formed in an amplification direction of the standing waves and in regions that correspond to anti-nodes of the standing waves.

The instantly soluble particle according to any one of <1> to <6>, and the method for producing an instantly soluble particle according to <7> or <8> can solve the conventionally existing problems and can achieve the object of the present disclosure.

1. An instantly soluble particle comprising:
a water-soluble base material; and
a poorly water-soluble compound,
wherein the water-soluble base material comprises a rapidly water-soluble compound, and
the poorly water-soluble compound exists in the water-soluble base material in an amorphous state.

2. The instantly soluble particle according to claim 1,
wherein the rapidly water-soluble compound is at least one selected from the group consisting of monosaccharides and disaccharides.

3. The instantly soluble particle according to claim 1,
wherein an amount of the poorly water-soluble compound is 75% by mass or less.

4. The instantly soluble particle according to claim 1,
wherein the amount of the poorly water-soluble compound is 10% by mass or more but 50% by mass or less.

5. The instantly soluble particle according to claim 1,
wherein a volume average particle diameter (Dv) of the instantly soluble particle is 0.5 μm or more but 50 μm or less.

6. The instantly soluble particle according to claim 1,
wherein the volume average particle diameter (Dv) of the instantly soluble particle is 0.5 μm or more but 20 μm or less.

7. A method for producing an instantly soluble particle, the method comprising:
discharging a liquid containing a rapidly water-soluble compound and a poorly water-soluble compound from a discharging hole to form liquid droplets; and
solidifying the liquid droplets.

8. The method for producing an instantly soluble particle according to claim 7,
wherein the discharging includes applying vibration to a liquid composition housed in a liquid-column-resonance liquid chamber to form standing waves through liquid column resonance and discharging the liquid composition from the discharging hole, the discharging hole being formed in an amplification direction of the standing waves and in regions that correspond to anti-nodes of the standing waves.
